(19) United States
(12) Patent Application Publication
(10) Pub. No.: US 2021/0280300 A1
(43) Pub. Date: Sep. 9, 2021

(54) MEDICAL INFORMATION PROCESSING SYSTEM, MEDICAL INFORMATION PROCESSING METHOD, AND STORAGE MEDIUM

(71) Applicant: CANON KABUSHIKI KAISHA, Tokyo (JP)

(72) Inventor: Manami Hikosaka, Kanagawa (JP)

(21) Appl. No.: 17/328,184

(22) Filed: May 24, 2021

Related U.S. Application Data
(63) Continuation of application No. PCT/JP2019/044802, filed on Nov. 15, 2019.

(30) Foreign Application Priority Data
Nov. 29, 2018 (JP) 2018-223614

(51) Int. Cl. G16H 30/40; G16H 30/20; G06F 16/28

(52) U.S. Cl. CPC G16H 30/40; G06F 16/285; G16H 30/20

(57) ABSTRACT

An image capturing control unit in a medical information processing apparatus acquires a medical image and related information, which are associated with each other, from a storage apparatus, such as a Picture Archiving and Communication Systems (PACS), in which the medical image obtained by capturing an image of a subject and the related information about the medical image are stored in association with each other, and the image capturing control unit limits an output of edited related information to the storage apparatus, in accordance with setting information set in the acquired related information.

### CROSS-REFERENCE TO RELATED APPLICATIONS

This application is a Continuation of International Patent Application No. PCT/JP2019/044802, filed Nov. 15, 2019, which claims the benefit of Japanese Patent Application No. 2018-223614, filed Nov. 29, 2018, both of which are hereby incorporated by reference herein in their entirety.

### BACKGROUND OF THE INVENTION

### Field of the Invention

The present invention relates to a medical information processing system, a medical information processing method, and a storage medium.

### Background Art

In recent years, there is a demand for assisting in reading a medical image, for example, as an additional role for healthcare professionals, such as a radiology technologist. For example, in a case where a radiologist or a specialist is absent during night-time or on a day when a hospital is closed, a radiology technologist who observes medical images on regular basis is expected to point out an abnormality.

For example, in a case where a radiology technologist informs a doctor of something noticeable during image capturing or something noticeable about a medical image, for example, comment information called a key object defined in Digital Imaging and Communications in Medicine (DICOM) standards can be used. This key object is related information about a medical image. For example, the key object is added to a medical image, and the key object and the medical image are output to an image server serving as a storage apparatus. The doctor can acquire the medical image to which the key object is added from the image server, and can read the medical image with reference to the comment information.

In regard to an image reading report that is related information to be added to a medical image, Japanese Patent Application Laid-Open No. 2004-334466 proposes a technique for adding an update history to manage creation or update of the report.

### CITATION LIST

### Patent Literature

PTL 1: Japanese Patent Laid-Open No. 2004-334466

However, in the technique discussed in Japanese Patent Application Laid-Open No. 2004-334466, if the related information stored in the storage apparatus is inadvertently updated, a trouble, such as misuse of the related information that is not suitable for intended use, or missing of permanent information such as calling for attention on the medical image, can occur. In other words, in the related art, it is difficult to prevent related information about a medical image from being inadvertently updated and to appropriately manage the related information.

The present invention has been made in view of the above-described issues, and is directed to providing a mechanism for preventing related information about a medical image from being inadvertently updated and for appropriately managing the related information.

### SUMMARY OF THE INVENTION

A medical information processing system according to the present invention includes a storage unit configured to store a medical image obtained by capturing an image of a subject and related information about the medical image in association with each other, and an editing control unit configured to limit editing including a change of the related information stored in the storage unit, in accordance with setting information set in the related information.

Another aspect of a medical information processing system according to the present invention includes a storage unit configured to store a medical image obtained by capturing an image of a subject and related information about the medical image in association with each other, an acquisition unit configured to acquire the medical image and the related information associated with each other, an editing unit configured to edit the related information acquired by the acquisition unit, and an output control unit configured to limit an output of the edited related information to the storage unit, in accordance with setting information set in the related information.

Further, the present invention includes a medical information processing method that is performed by the medical information processing system described above, and a storage medium storing a program for causing a computer to execute the medical information processing method.

Further features of the present invention will become apparent from the following description of exemplary embodiments with reference to the attached drawings.

### BRIEF DESCRIPTION OF THE DRAWINGS

FIG. 1 is a diagram illustrating an example of a schematic configuration of a medical information processing system according to a first exemplary embodiment of the present invention.

FIG. 2A is a diagram illustrating an example of a new examination input screen displayed on a display unit illustrated in FIG. 1 according to the first exemplary embodiment of the present invention.

FIG. 2B is a diagram illustrating an example of a new examination input screen displayed on a display unit illustrated in FIG. 1 according to the first exemplary embodiment of the present invention.

FIG. 3 is a diagram illustrating an example of an image capturing information input screen displayed on the display unit illustrated in FIG. 1 according to the first exemplary embodiment of the present invention.

FIG. 4A is a diagram illustrating an example of an image capturing screen displayed on the display unit illustrated in FIG. 1 according to the first exemplary embodiment of the present invention.

FIG. 4B is a diagram illustrating an example of an image capturing screen displayed on the display unit illustrated in FIG. 1 according to the first exemplary embodiment of the present invention.

FIG. 5 is a diagram illustrating an example of a key object creation screen displayed on the display unit illustrated in FIG. 1 according to the first exemplary embodiment of the present invention.

FIG. 6A is a diagram illustrating an example of a key object created by a radiographic image capturing control apparatus (e.g., image capturing control unit) illustrated in FIG. 1 according to the first exemplary embodiment of the present invention.

FIG. 6B is a diagram illustrating an example of a key object created by a radiographic image capturing control apparatus (e.g., image capturing control unit) illustrated in FIG. 1 according to the first exemplary embodiment of the present invention.

FIG. 6C is a diagram illustrating an example of a key object created by a radiographic image capturing control apparatus (e.g., image capturing control unit) illustrated in FIG. 1 according to the first exemplary embodiment of the present invention.

FIG. 7 is a diagram illustrating an example of a past examination selection screen displayed on the display unit illustrated in FIG. 1 according to the first exemplary embodiment of the present invention.

FIG. 8 is a diagram illustrating an example of a past examination playback screen displayed on the display unit illustrated in FIG. 1 according to the first exemplary embodiment of the present invention.

FIG. 9 is a flowchart illustrating an example of a processing procedure for a medical information processing method that is performed by a medical information processing apparatus, according to the first exemplary embodiment of the present invention.

FIG. 10 is a flowchart illustrating an example of a processing procedure for a medical information processing method that is performed by a medical information processing apparatus, according to a second exemplary embodiment of the present invention.

### DESCRIPTION OF THE EMBODIMENTS

Modes (exemplary embodiments) for implementing the present invention will be described below with reference to the drawings.

### First Exemplary Embodiment

A description will be given of a first exemplary embodiment of the present invention.

<Configuration of Medical Information Processing System>

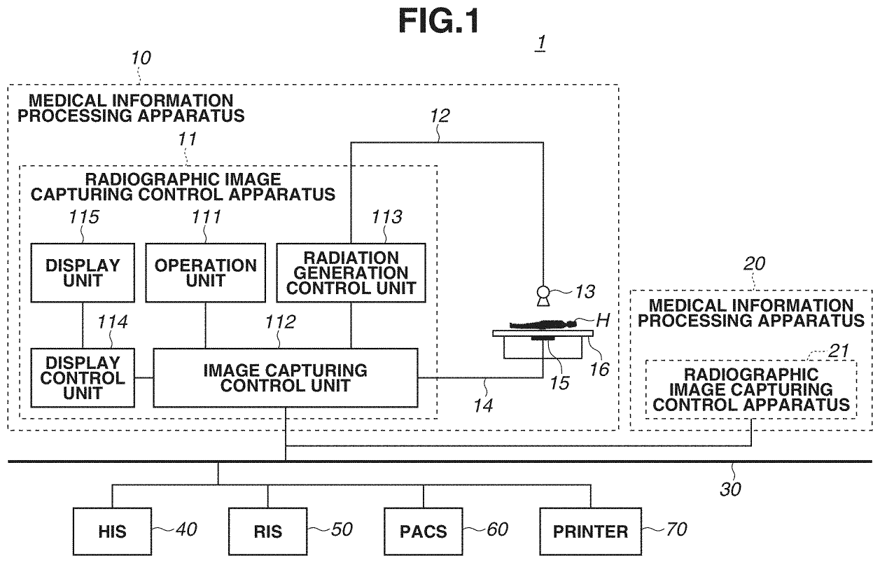

FIG. 1 is a diagram illustrating an example of a schematic configuration of a medical information processing system 1 according to a first exemplary embodiment of the present invention. The medical information processing system 1 includes a medical information processing apparatus 10 according to the present exemplary embodiment, another medical information processing apparatus 20, a network 30, a hospital information system (HIS) 40, a radiology information system (RIS) 50, a Picture Archiving and Communication Systems (PACS) 60, and a printer 70.

The medical information processing apparatus 10 is an apparatus that associates a medical image obtained by capturing an image of a subject H with related information about the medical image, and processes the medical image and the related information as medical information. The present exemplary embodiment will be described by taking a case where a radiological image obtained by capturing an image of the subject H using a ray of radiation, such as an X-ray, is applied as a “medical image”, as an example. However, the present invention is not limited to this example, and other medical images can also be applied. Further, the present exemplary embodiment illustrates will be described by taking a case where the above-described key object is applied as “related information about a medical image”, as an example. However, the present invention is not limited to this example, and other related information can also be applied.

The medical information processing apparatus 10 is an apparatus provided in an image capturing room. As illustrated in FIG. 1, the medical information processing apparatus 10 includes a radiographic image capturing control apparatus 11, a cable 12, a radiation generation apparatus 13, a cable 14, a radiation detection apparatus 15, and an image capturing platform 16. The components (11 to 16) of the medical information processing apparatus 10 will be described below.

The other medical information processing apparatus 20 is an apparatus provided in an image capturing room that is different from the image capturing room in which the medical information processing apparatus 10 is provided, and includes a radiographic image capturing control apparatus 21 corresponding to the radiographic image capturing control apparatus 11 of the medical information processing apparatus 10. In the present exemplary embodiment, the other medical information processing apparatus 20 can further include components respectively corresponding to the cable 12, the radiation generation apparatus 13, the cable 14, the radiation detection apparatus 15, and the image capturing platform 16 of the medical information processing apparatus 10.

The HIS 40 is namely an in-hospital information system that manages progress of radiographic image capturing. The RIS 50 is namely a radiation department information system. The PACS 60 is an image server serving as a storage apparatus that stores medical information. The printer 70 is an apparatus that prints out data.

The HIS 40 may include a server that manages accounting information. In the medical information processing system 1, in a case where it is determined that radiographic image capturing is required, an examination instruction is input from the HIS 40 and the examination instruction is transmitted to the radiation department that receives the examination instruction. This request information is referred to as an examination order. This examination order includes a name of the department that has requested an examination, examination items, and patient personal data. In a case where the RIS 50 receives an examination order, the radiation department adds image capturing conditions and the like to the examination order and transfers the examination order to, for example, the medical information processing apparatus 10. The medical information processing apparatus 10 executes radiographic image capturing in accordance with the received examination order. Then, the medical information processing apparatus 10 adds a key object, which is related information about the radiological image, and the like to a radiological image obtained by image capturing, and outputs the radiological image to the PACS 60 and the printer 70. Examination implementation information on the medical information processing apparatus 10 is transferred to the HIS 40. The implementation information transferred to the HIS 40 is used for management of the progress of an examination and also for accounting processing after the examination.

The network 30 interconnects the medical information processing apparatus 10, the other medical information processing apparatus 20, the HIS 40, the RIS 50, the PACS 60, and the printer 70 to allow communications with each other. The network 30 includes, for example, a local area network (LAN) or a wide area network (WAN). Each of the medical information processing apparatus 10, the other medical information processing apparatus 20, the HIS 40, the RIS 50, the PACS 60, and the printer 70, which are connected to each other via the network 30, includes, for example, one or more computers. In this case, each computer included in the components includes, for example, a main control unit, such as a central processing unit (CPU), and a storage unit, such as a read-only memory (ROM) or a random access memory (RAM). Each computer may further include a communication unit, such as a network card, and an input/output unit, such as a keyboard, a display, or a touch panel. These components of each computer are connected to each other with a bus or the like. The main control unit reads out and executes programs stored in the storage unit, to control the components.

<Configuration of Medical Information Processing Apparatus>

Next, a schematic configuration of the medical information processing apparatus 10 illustrated in FIG. 1 will be described.

As described above, the medical information processing apparatus 10 includes the radiographic image capturing control apparatus 11, the cable 12, the radiation generation apparatus 13, the cable 14, the radiation detection apparatus 15, and the image capturing platform 16.

The radiographic image capturing control apparatus 11 controls the operation of the medical information processing apparatus 10 in an integrated manner, and performs various processing. As illustrated in FIG. 1, the radiographic image capturing control apparatus 11 includes an operation unit 111, an image capturing control unit 112, a radiation generation control unit 113, a display control unit 114, and a display unit 115.

The operation unit Ill inputs information input by an operator operating the operation unit 111 to the image capturing control unit 112. The operation unit 111 includes, for example, a mouse and operation buttons.

The image capturing control unit 112 controls radiographic image capturing of the subject H in an integrated manner, for example, based on the information input from the operation unit 111, and performs various processing.

The radiation generation control unit 113 is connected to the radiation generation apparatus 13 via the cable 12, and controls irradiation of a ray of radiation generated from the radiation generation apparatus 13, based on control of the image capturing control unit 112. The radiation generation apparatus 13 includes, for example, a radiation vessel, and irradiates a ray of radiation toward the subject H (e.g., a specific region of a patient), based on control of the radiation generation control unit 113. In the example illustrated in FIG. 1, the image capturing platform 16 that supports the subject H is placed. The image capturing platform 16 is configured to support the subject H in a state where the subject H is erect or lying.

Further, the image capturing control unit 112 is connected to the radiation detection apparatus 15 via the cable 14. Power, a radiological image signal, a control signal, and the like are delivered between the image capturing control unit 112 and the radiation detection apparatus 15 via the cable 14. The radiation detection apparatus 15 is a detection apparatus that detects a ray of radiation that has passed through the subject H (further the image capturing platform 16) as an electrical signal, based on control of the image capturing control unit 112, and acquires a radiological image (radiological image signal). Specifically, the radiation generation apparatus 13 and the radiation detection apparatus 15 operate in a cooperative manner, to implement a radiographic image capturing apparatus. The radiation detection apparatus 15 is placed at a predetermined position on the image capturing platform 16.

The image capturing control unit 112 also functions as an instruction unit that instructs to execute starting of radiographic image capturing corresponding to at least one of examination orders received from the RIS 50. Each examination order includes, for example, information about a subject, i.e., a patient, and image capturing condition information including one or more image capturing regions of the subject. Starting of radiographic image capturing may be executed, for example, also in a case where the image capturing control unit 112 selects an examination order based on which image capturing is executed, other than in a case where an instruction is input from the operator via the operation unit 111.

The display control unit 114 controls display of various images (including a radiological image) and various information, based on control of the image capturing control unit 112.

The display unit 115 is composed of, for example, a liquid crystal display, and displays various images and various information to the operator (e.g., an image capturing engineer or a doctor), based on control of the display control unit 114. The display unit 115 and the operation unit 111 may be integrated together and implemented as a touch panel. In the medical information processing apparatus 10, in a case where radiographic image capturing is executed, for example, a radiological image obtained by radiographic image capturing is displayed on the display unit 115. Then, the operator can perform image processing and image editing, such as clipping, addition of an annotation, and geometric transform, on the radiological image displayed on the display unit 115 via the operation unit 111.

The configuration illustrated in FIG. 1 is merely an example and can be changed as needed. For example, in FIG. 1, various apparatuses and systems are connected to the radiographic image capturing control apparatus 11 via the network 30. However, the radiographic image capturing control apparatus 11 can be disconnected from such apparatuses or systems. For example, medical information including a radiological image for diagnosis may be output and stored in a portable storage medium, such as a digital versatile disc (DVD), and the medical information may be input to various apparatuses and systems via the portable storage medium. The network 30 may be configured with a wire. Apart of the network 30 may be configured using a wireless signal transmission path. The various apparatuses and systems may be provided on the network 30 as a plurality of apparatuses and systems. For example, in a case where a plurality of image capturing rooms is present in a hospital, a plurality of medical information processing apparatuses 10 and 20 may be present.

<Image Capturing Processing>

Next, a processing procedure for capturing a radiological image according to an examination procedure of the medical information processing system 1 illustrated in FIG. 1 will be described.

First, patient information and examination information are input to the medical information processing apparatus 10, based on an examination request form or an examination order received from the RIS 50. The patient information includes information, such as a patient name and a patient identification (ID), and the examination information includes image capturing information in which contents of image capturing to be executed on a patient are prescribed.

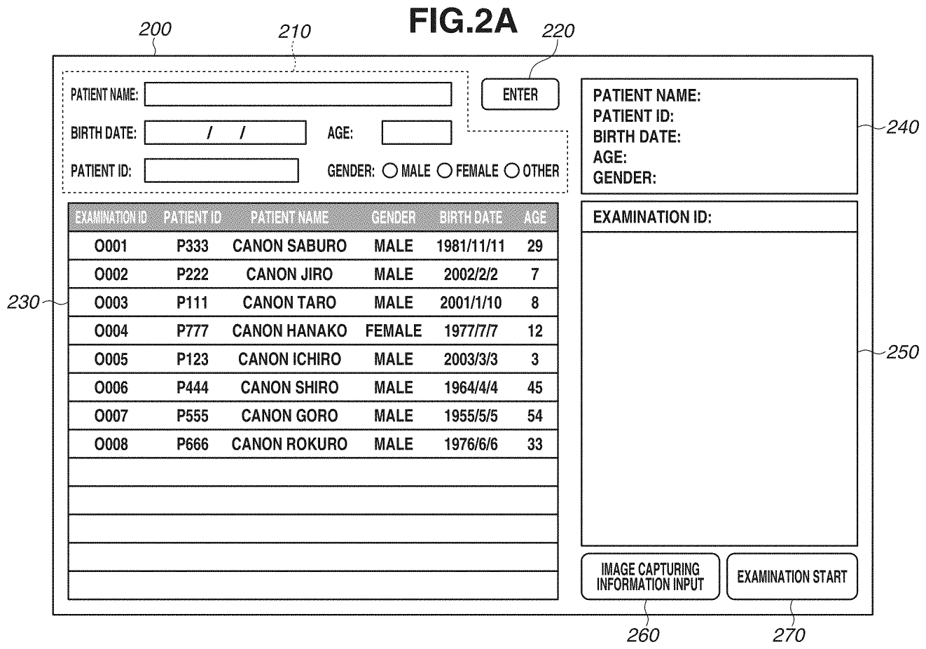

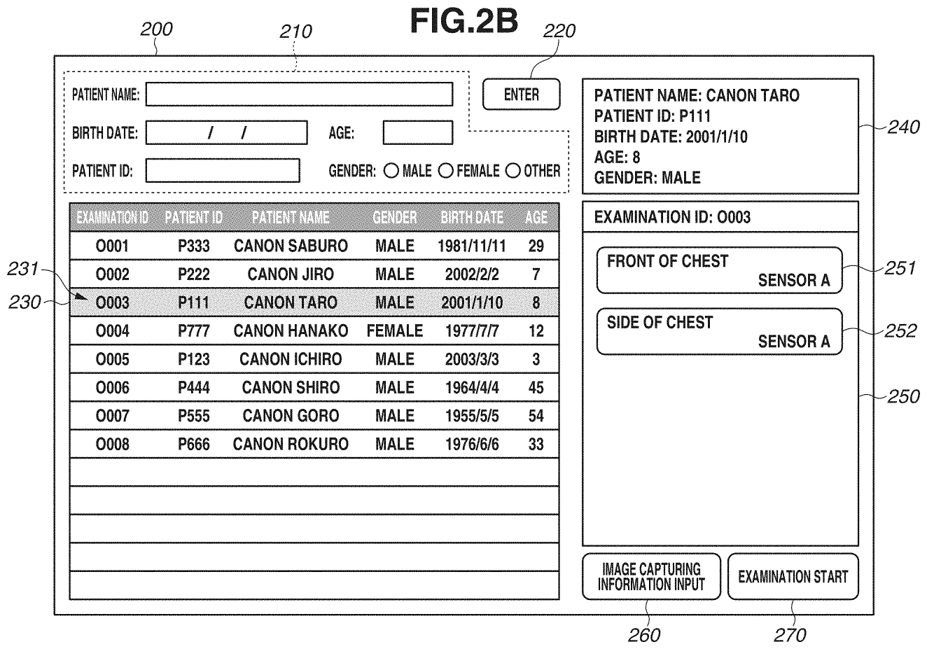

Next, the medical information processing apparatus 10 displays a new examination input screen on the display unit 115, based on control of the display control unit 114. FIGS. 2A and 2B are diagrams each illustrating an example of a new examination input screen 200 displayed on the display unit 115 illustrated in FIG. 1 according to the first exemplary embodiment of the present invention.

The new examination input screen 200 illustrated in FIG. 2A includes a patient information input area 210, a patient information enter button 220, a request examination list (examination order list) display area 230, a patient information display area 240, an image capturing information display area 250, an image capturing information input button 260, and an examination start button 270.

The request examination list display area 230 displays a list of examination orders received from the RIS 50. Next, as illustrated in FIG. 2B, for example, in a case where the operator selects an examination 231 from the request examination list displayed on the request examination list display area 230 via the operation unit 111, patient information (patient ID, patient name, birth date, etc.) of a patient corresponding to the selected examination 231 is displayed on the patient information display area 240. Further, an examination ID of the selected examination 231 is displayed on the image capturing information display area 250 illustrated in FIG. 2B, and image capturing information corresponding to the examination ID is displayed on an area immediately below the area on which the examination ID is displayed. This image capturing information is received from the RIS 50 as described above. In the example illustrated in FIG. 2B, a “front of chest” button 251 and a “side of chest” button 252, which are image capturing method buttons corresponding to the image capturing information, are arranged.

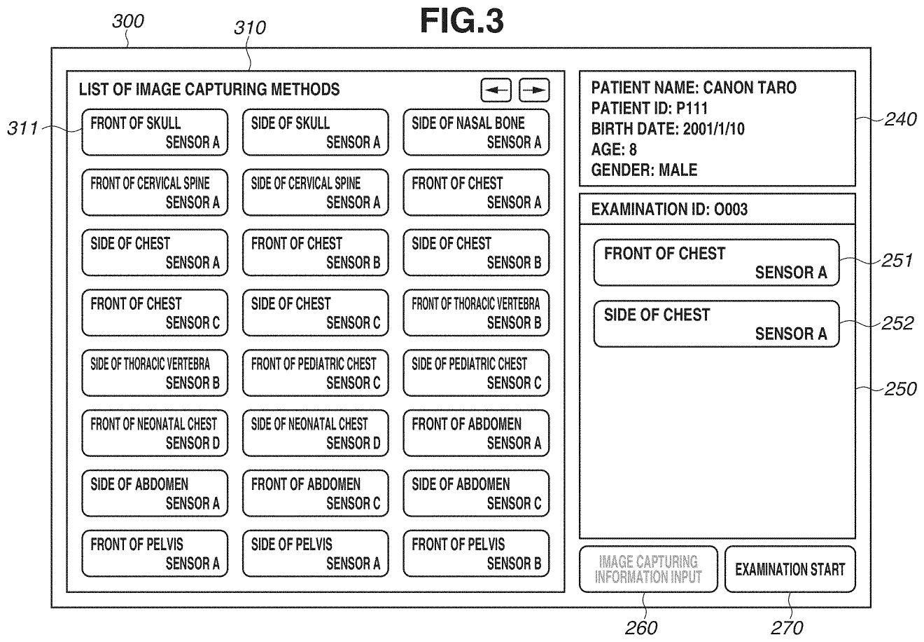

For example, in a case where the operator presses the image capturing information input button 260 illustrated in FIGS. 2A and 2B via the operation unit 111, the medical information processing apparatus 10 displays an image capturing information input screen on the display unit 115, based on control of the display control unit 114. FIG. 3 is a diagram illustrating an example of an image capturing information input screen 300 displayed on the display unit 115 illustrated in FIG. 1 according to the first exemplary embodiment of the present invention. In FIG. 3, components similar to those illustrated in FIGS. 2A and 2B are denoted by the same reference numerals and detailed descriptions thereof are omitted.

The image capturing information input screen 300 illustrated in FIG. 3 has a configuration in which an image capturing information input area 310 is arranged in the portion corresponding to the patient information input area 210, the patient information enter button 220, and the request examination list display area 230 of the new examination input screen 200 illustrated in FIGS. 2A and 2B. In the example of the image capturing information input screen 300 illustrated in FIG. 3, a plurality of image capturing method selection buttons 311 is displayed on the image capturing information input area 310, and an image capturing method can be added by selecting a button from among the image capturing method selection buttons 311. The added image capturing method is displayed together with the “front of chest” button 251 and the “side of chest” button 252 on the image capturing information display area 250. Each image capturing method is associated with an image capturing method ID.

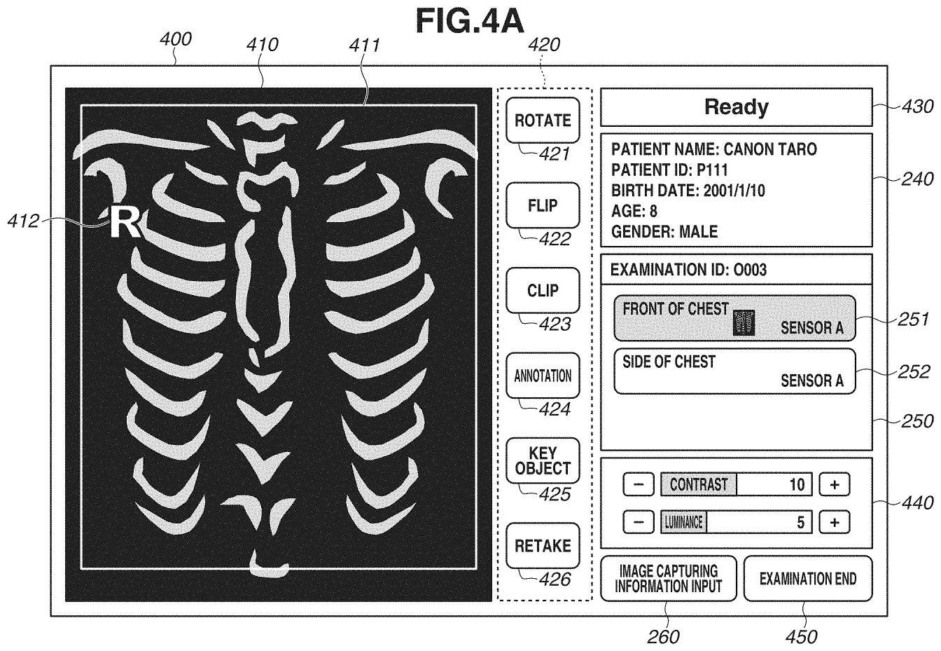

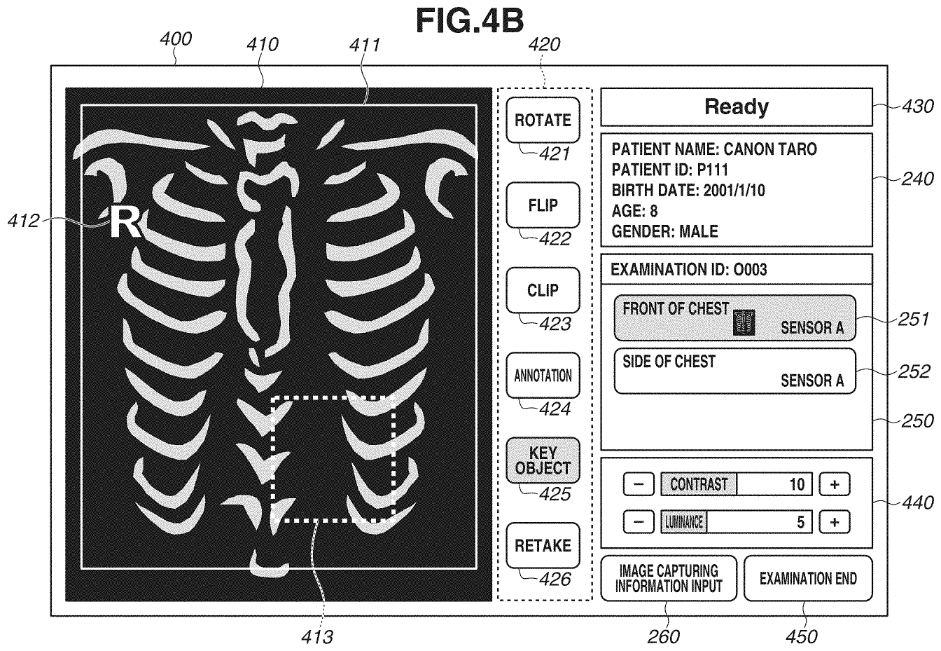

After checking the patient information and the image capturing information displayed on the display unit 115, for example, in a case where the operator presses the examination start button 270 via the operation unit 111, the medical information processing apparatus 10 enters the examination to be executed. Then, the medical information processing apparatus 10 displays an image capturing screen on the display unit 115 in response to pressing of the examination start button 270, based on control of the display control unit 114. FIGS. 4A and 4B are diagrams each illustrating an image capturing screen 400 displayed on the display unit 115 illustrated in FIG. 1 according to the first exemplary embodiment of the present invention. The image capturing screen 400 illustrated in FIGS. 4A and 4B is a screen that is used during image capturing, and components similar to those illustrated in FIGS. 2A and 2B are denoted by the same reference numerals.

On the image capturing screen 400 illustrated in FIG. 4A, an image display area 410, an image operation button display area 420, a message display area 430, the patient information display area 240, the image capturing information display area 250, an image processing setting area 440, the image capturing information input button 260, and an examination end button 450 are provided. On the image operation button display area 420, a rotate button 421, a flip button 422, a clip button 423, an annotation button 424, a key object button 425, and a retake button 426 are provided.

In a case where the image capturing screen 400 illustrated in FIG. 4A is displayed on the display unit 115, the “front of chest” button 251, which is an image capturing method button disposed at an uppermost portion in the image capturing information display area 250, is selected in default. Accordingly, the image capturing control unit 112 of the medical information processing apparatus 10 transmits image capturing conditions (tube voltage or tube current of the radiation generation apparatus 13, a radiation irradiation time, etc.) set corresponding to the image capturing method button (image capturing method) to the radiation generation control unit 113. At the same time, the image capturing control unit 112 controls the radiation detection apparatus 15 according to the image capturing conditions, to prepare for radiographic image capturing. When the preparation is ready, the medical information processing apparatus 10 shifts to an image capturing ready state. In this case, a message “Ready” indicating the image capturing ready state is displayed on the message display area 430.

Next, after checking the image capturing method displayed on the display unit 115, the operator makes settings for image capturing and positions the patient, to complete a series of image capturing preparation. Next, after checking the image capturing ready state with reference to the message display area 430, in a case where the operator presses a radiation irradiation switch, which is a component of the operation unit 111, the image capturing control unit 112 detects the pressing. Upon detecting the pressing of the radiation irradiation switch, the image capturing control unit 112 causes the radiation generation apparatus 13 to irradiate a ray of radiation toward the subject H (e.g., a specific region of the patient) via the radiation generation control unit 113, and causes the radiation detection apparatus 15 to detect the ray of radiation that has transmitted the subject H (further the image capturing platform 16). Thus, a radiological image of the subject H is captured.

Upon completion of the image capturing, the image capturing control unit 112 acquires the radiological image (radiological image signal) from the radiation detection apparatus 15, and executes image processing on the acquired radiological image, based on a predetermined image processing condition. The predetermined image processing condition is prescribed to conform to, for example, an image capturing method. When the image processing is finished, the image capturing control unit 112 causes the radiological image subjected to the image processing to be displayed on the image display area 410 via the display control unit 114.

In a case where the operator changes contrast or the like of the radiological image displayed on the image display area 410, the operator operates, for example, contrast and luminance buttons provided on the image processing setting area 440. In a case where the operator changes an area to be clipped from an output image, the operator operates various adjustment buttons provided on the image operation button display area 420. In a case of clipping a part of an image, the operator operates the clip button 423, a clip box 411, or the like, to designate a desired clip area. In a case of adding a character string as diagnosis information, the operator operates the annotation button 424 or the like to superimpose a character string indicated by an annotation 412 on the radiological image. In a case where an orientation of the radiological image is not suitable for diagnosis, the operator performs geometric transform using the rotate button 421, the flip button 422, or the like. Thus, the operator can execute additional image editing on the radiological image displayed on the image display area 410. Further, for example, in a case of creating a key object as related information about the medical image (radiological image), the operator designates a key object box 413 illustrated in FIG. 4B and operates the key object button 425 or the like.

The operator executes all image capturing operations corresponding to image capturing methods displayed in the image capturing information display area 250 by repeating the above-described procedure. After all the image capturing operations are finished, the operator presses the examination end button 450. Thus, a series of examination is finished, and the radiographic image capturing control apparatus 11 displays the new examination input screen 200 illustrated in FIGS. 2A and 2B again on the display unit 115. In this operation, the radiographic image capturing control apparatus 11 adds additional information, such as examination information about a radiological image that is a retake target and is not used, and image capturing conditions, to the radiological image in the image capturing control unit 112, and the radiological image is output to, for example, the PACS 60, the printer 70, or the storage unit, such as a ROM, of the radiographic image capturing control apparatus 11. In the storage unit, such as the RIS 50 or the ROM, the radiological image and the patient information are stored in association with each other.

<Key Object Creation Processing>

Next, a processing procedure for creating a key object as related information about a medical image (radiological image) according to an examination procedure of the medical information processing system 1 illustrated in FIG. 1 will be described.

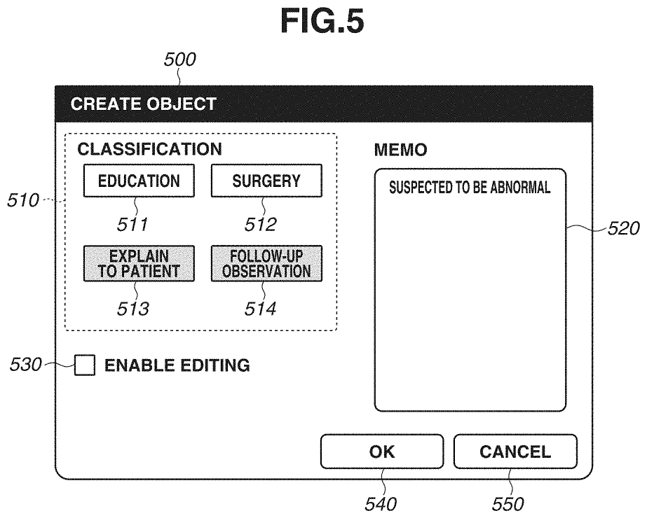

The operator checks the captured radiological image on the image capturing screen 400 illustrated in FIGS. 4A and 4B, and then issues an instruction to create a key object, as needed, via the operation unit 111. Specifically, for example, as illustrated in FIG. 4B, the operator presses the key object button 425 and operates the key object box 413 to designate a desired target area. In a case where the key object button 425 is pressed and the target area is designated, the medical information processing apparatus 10 displays a key object creation screen on the display unit 115, based on control of the display control unit 114. FIG. 5 is a diagram illustrating an example of a key object creation screen 500 displayed on the display unit 115 illustrated in FIG. 1 according to the first exemplary embodiment of the present invention.

The key object creation screen 500 illustrated in FIG. 5 includes a classification setting unit 510, an annotation designation unit 520, an editing enable/disable setting unit 530, an OK button 540, and a cancel button 550.

The operator operates the classification setting unit 510 to set an intended use of the key object. In the classification setting unit 510, various buttons are arranged for classifications each indicating an intended use of the key object. Specifically, in the example illustrated in FIG. 5, the classification setting unit 510 is provided with an education button 511, a surgery button 512, an explain-to-patient button 513, and a follow-up observation button 514. For example, in a case where the key object to be created is used for explanation to a patient and for follow-up observation, the operator presses the explain-to-patient button 513 and the follow-up observation button 514 as illustrated in FIG. 5, and changes the state of each button to a selected state.

Further, the operator writes a memo, such as an explanation about the key object, and inputs the memo to the annotation designation unit 520. In this operation, for example, in a case where there is no need to explain the key object and the above-described classification is sufficient, the operator need not necessarily input the memo.

Further, the operator sets, in the editing enable/disable setting unit 530, information indicating whether the key object that is being created can be edited after the key object is created. In a case where the key object that is being created is permanent information about a medical image (radiological image), the operator turns off the editing enable/disable setting unit 530 to prohibit editing (editing disabled) after creation. Further, for example, in a case where the key object is information that is not required after the key object that is being created is checked, or is information that is effective only for a certain period, the operator turns on the editing enable/disable setting unit 530 to enable editing after creation (editing enabled).

After setting information necessary for the key object, in response to the operator pressing the OK button 540, for example, the image capturing control unit 112 creates a key object, based on information set on the key object creation screen 500, and acquires the key object. Further, for example, the image capturing control unit 112 adds the acquired key object to the radiological image displayed on the image display area 410 of the image capturing screen 400 illustrated in FIGS. 4A and 4B, and associates the radiological image with the key object.

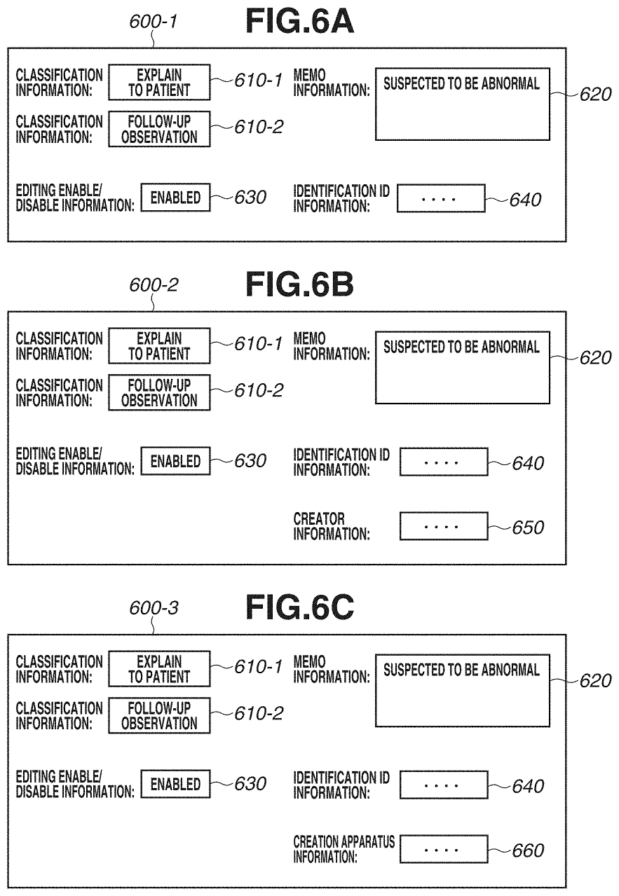

FIGS. 6A, 6B, and 6C are diagrams each illustrating an example of a key object 600 created by the radiographic image capturing control apparatus 11 (e.g., image capturing control unit 112) illustrated in FIG. 1 according to the first exemplary embodiment of the present invention. Specifically, an example of the key object created based on the key object creation screen 500 illustrated in FIG. 5 is a key object 600-1 illustrated in FIG. 6A. The key object 600-1 illustrated in FIG. 6A includes explain-to-patient classification information 610-1 and follow-up observation classification information 610-2 set in the classification setting unit 510 illustrated in FIG. 5 as classification information 610. The key object 600-1 illustrated in FIG. 6 includes memo information 620 set in the annotation designation unit 520 illustrated in FIG. 5, and editing enable/disable information 630 set in the editing enable/disable setting unit 530 illustrated in FIG. 5. In the example illustrated in FIG. 6A, the editing enable/disable setting unit 530 is turned on and the editing enable/disable information 630 indicating that editing is enabled is displayed. The key object 600-1 illustrated in FIG. 6A further includes identification ID information 640 for associating the key object 600-1 with the medical image (radiological image). Specifically, in the key object 600-1 illustrated in FIG. 6A, the classification information 610, the memo information 620, the editing enable/disable information 630, and the identification ID information 640 are displayed as setting information set in the key object 600-1.

In a case where the operator presses the cancel button 550 illustrated in FIG. 5, for example, the image capturing control unit 112 discards the key object that is being created. Further, in a case where the OK button 540 and the cancel button 550 illustrated in FIG. 5 are pressed, the medical information processing apparatus 10 closes the key object creation screen 500 illustrated in FIG. 5, based on control of the display control unit 114, and displays the image capturing screen 400 illustrated in FIGS. 4A and 4B on the display unit 115.

After the key object is created, for example, in response to the operator pressing the examination end button 450 on the image capturing screen 400 illustrated in FIGS. 4A and 4B, the image capturing control unit 112 adds and associates the created key object as related information about the medical image (radiological image), and then outputs the related information to, for example, the PACS 60, the printer 70, or the storage unit, such as a ROM, of the radiographic image capturing control apparatus 11.

As described above, the medical image (radiological image) and the related information (key object) about the medical image are output to the PACS 60 and the like from the medical information processing apparatus 10, and the output medical image and related information are stored in association with each other in the PACS 60 or the like. The above-described example illustrates a case where the medical image and the related information generated by the medical information processing apparatus 10 are stored in the PACS 60 or the like. However, the present invention is not limited to this mode. For example, the medical image (radiological image) and the related information (key object) may be output from the other medical information processing apparatus 20 having a functional configuration similar to that of the medical information processing apparatus 10, and the output medical image and related information may be stored in association with each other in the PACS 60 or the like.

<Processing for Re-Outputting Key Object to Storage Apparatus>

Next, a processing procedure for re-outputting a key object to the PACS 60, which is a storage apparatus, or the like, according to an examination procedure of the medical information processing system 1 illustrated in FIG. 1 will be described. As processing for re-outputting the key object, the present exemplary embodiment illustrates an example where the medical image (radiological image) and the related information (key object) that are output from the medical information processing apparatus 10 or the other medical information processing apparatus 20 and are stored in the PACS 60 or the like are acquired by the medical information processing apparatus 10, and the re-output of the key object to the storage apparatus, such as the PACS 60, is limited in accordance with setting information set as the acquired key object.

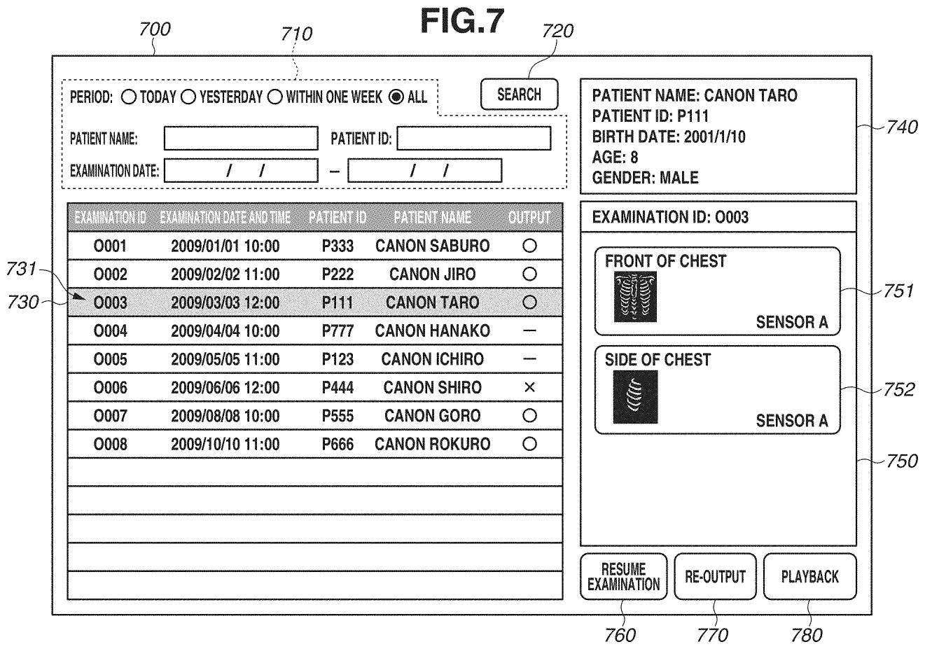

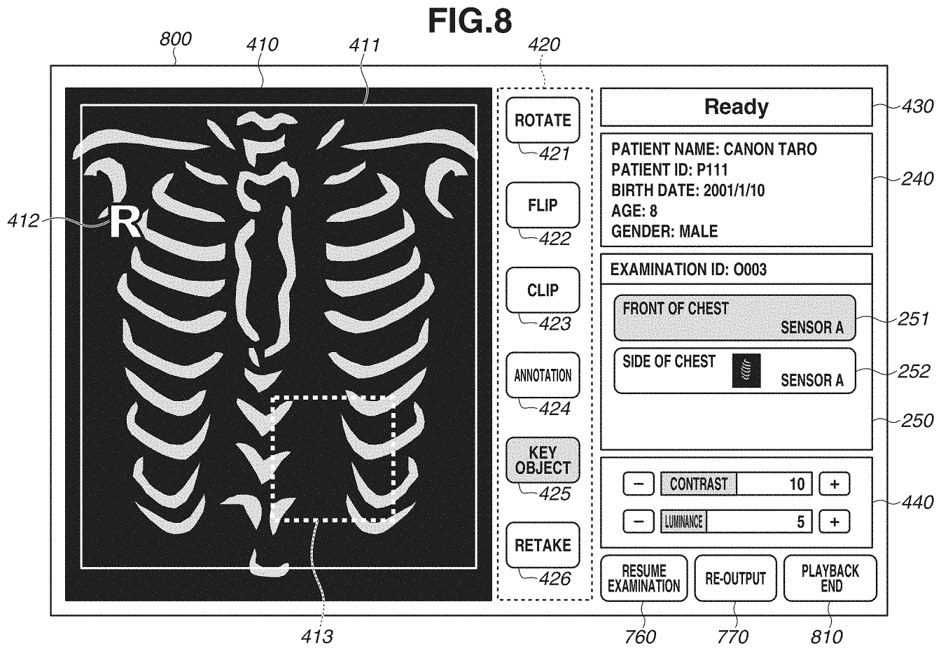

In a case where key object re-output processing is performed on the PACS 60 or the like, the medical information processing apparatus 10 first displays a past examination selection screen on the display unit 115, based on control of the display control unit 114. FIG. 7 is a diagram illustrating an example of a past examination selection screen 700 displayed on the display unit 115 illustrated in FIG. 1 according to the first exemplary embodiment of the present invention.

The past examination selection screen 700 illustrated in FIG. 7 includes a search condition input area 710, a search start button 720, a past examination list display area 730, a patient information display area 740, an image capturing information display area 750, an examination resume button 760, an examination re-output button 770, and an examination playback button 780.

On the past examination list display area 730, a list of examinations (examinations including medical information about the medical image (radiological image) and related information (key object)) executed by the radiographic image capturing control apparatus 11 is displayed. In addition, an examination executed by another radiographic image capturing control apparatus 21 on the network 30, an examination including medical information held in the PACS 60, and the like may also be displayed.

For example, in response to the operator selecting an examination 731 from the past examination list displayed on the past examination list display area 730 via the operation unit 111, patient information (patient ID, patient name, birth date, etc.) corresponding to a patient of the selected examination 731 is displayed on the patient information display area 740. Further, on the image capturing information display area 750, an examination ID for the selected examination 731 is displayed and information about image capturing executed in the selected examination 731 is displayed on an area immediately below the area on which the examination ID is displayed. In the example illustrated in FIG. 7, a “front of chest” button 751 and a “side of chest” button 752, each of which is an image capturing method button including a radiological image obtained by executed image capturing, are arranged.

On the past examination selection screen 700 illustrated in FIG. 7, for example, in response to the operator pressing the examination resume button 760 via the operation unit 111, the state of the executed examination can be changed into a suspended state and an image capturing method can be added.

Further, on the past examination selection screen 700 illustrated in FIG. 7, for example, in response to the operator pressing the examination playback button 780 via the operation unit 111, the medical information processing apparatus 10 displays a past examination playback screen of the selected examination 731 on the display unit 115, based on control of the display control unit 114. FIG. 8 is a diagram illustrating an example of a past examination playback screen 800 displayed on the display unit 115 illustrated in FIG. 1 according to the first exemplary embodiment of the present invention. In FIG. 8, components similar to those illustrated in FIGS. 4A and 4B and FIG. 7 are denoted by the same reference numerals and detailed descriptions thereof are omitted.

On the past examination playback screen 800 illustrated in FIG. 8 includes the image display area 410, the image operation button display area 420, the message display area 430, the patient information display area 240, the image capturing information display area 250, the image processing setting area 440, the examination resume button 760, the examination re-output button 770, and a playback end button 810. The past examination playback screen 800 basically includes a display area corresponding to the image capturing screen 400 illustrated in FIG. 4 and the past examination selection screen 700 illustrated in FIG. 7. The difference in the display area from the image capturing screen 400 illustrated in FIGS. 4A and 4B is that, as illustrated in FIG. 8, the image capturing information input button 260 and the examination end button 450 illustrated in FIGS. 4A and 4B are not displayed and the examination resume button 760, and the examination re-output button 770 and the playback end button 810 illustrated in FIG. 7 are newly displayed.

On the past examination playback screen 800 illustrated in FIG. 8, for example, in response to the operator pressing the key object button 425 via the operation unit 111, the medical information processing apparatus 10 displays the key object creation screen 500 illustrated in FIG. 5 on the display unit 115, based on control of the display control unit 114. The key object (e.g., the created key object 600-1 illustrated in FIG. 6A) created in the examination may be held on the key object creation screen 500, and the set key object may be edited by an operation on the key object creation screen 500. For example, in response to the operator pressing the examination re-output button 770 via the operation unit 111, the medical information processing apparatus 10 re-outputs the examination that is already output to an external apparatus such as the PACS 60. Objects included in the examination to be re-output include not only the radiological image obtained by image capturing and the examination information, and also additional information, such as the key object that is related information. In a case where re-output to the PACS 60 or the like is executed, the medical information processing apparatus 10 determines whether re-output is enabled (in particular, whether re-output of the key object is enabled), and limits the re-output, as needed. After all operations required on the past examination playback screen 800 are finished, the operator presses the playback end button 810. Thus, playback of the past examination is finished, and the radiographic image capturing control apparatus 11 displays the past examination selection screen 700 illustrated in FIG. 7 again on the display unit 115.

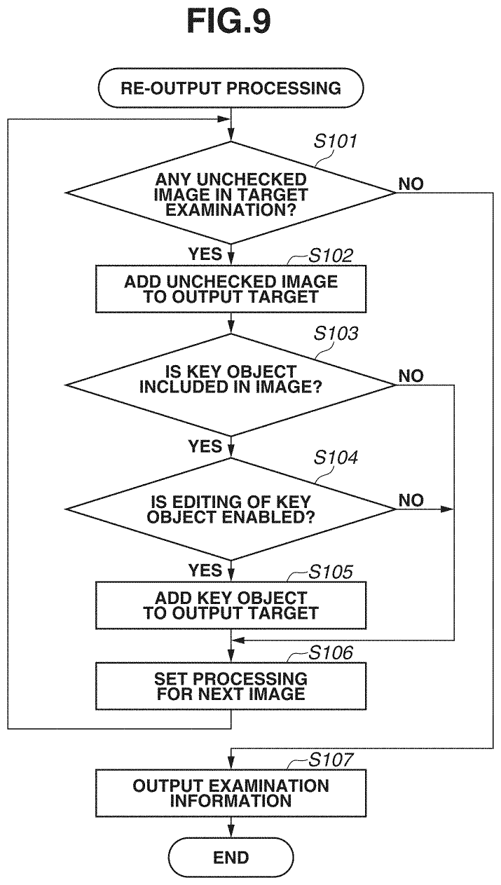

FIG. 9 is a flowchart illustrating an example of a processing procedure for a medical information processing method that is performed by the medical information processing apparatus 10 according to the first exemplary embodiment of the present invention. Specifically, FIG. 9 is a flowchart illustrating an example of a processing procedure for key object re-output processing on the PACS 60, which is a storage apparatus, or the like in the medical information processing apparatus 10.

Prior to the processing in the flowchart illustrated in FIG. 9, the image capturing control unit 112 first performs processing for acquiring various information about the examination targeted for re-output processing from the PACS 60, which is a storage apparatus, or the like. Specifically, in this case, the image capturing control unit 112 preliminarily acquires information about the examination, including the radiological image and the key object, targeted for re-output processing from the PACS 60 or the like in which the radiological image, which is a medical image obtained by capturing an image of the subject H, and the key object, which is related information about the radiological image, are stored in association with each other. The image capturing control unit 112 that performs this processing constitutes an acquisition unit.

In a case where the processing in the flowchart illustrated in FIG. 9 is started, first, in step S101, for example, the image capturing control unit 112 determines whether there is any unchecked radiological image in the examination targeted for re-output processing.

As a result of determination in step S101, in a case where there is an unchecked radiological image in the examination targeted for re-output processing (YES in step S101), the processing proceeds to step S102.

In step S102, for example, the image capturing control unit 112 adds the unchecked radiological image to an output target.

Next, in step S103, for example, the image capturing control unit 112 determines whether the key object is added to the radiological image added to the output target in step S102.

As a result of determination in step S103, in a case where the key object is added to the radiological image added to the output target (YES in step S103), the processing proceeds to step S104.

In step S104, for example, the image capturing control unit 112 determines whether editing of the key object added to the radiological image added to the output target is enabled at creation of the key object. Specifically, for example, in a case where the key object added to the radiological image added to the output target corresponds to the key object 600-1 illustrated in FIG. 6A, the determination in step S104 is performed with reference to the editing enable/disable information 630. In the case of the key object 600-1 illustrated in FIG. 6A, “enable” is set as the editing enable/disable information 630, and thus the determination result is “YES” in step S104.

As a result of determination in step S104, in a case where editing of the key object added to the radiological image added to the output target is enabled (YES in step S104), the processing proceeds to step S105.

In step S105, for example, the image capturing control unit 112 adds the key object to the output target.

In a case where the processing of step S105 is finished, in a case where the determination result in step S103 is “NO” (NO in step S103), or in a case where the determination result in step S104 is “NO” (NO in step S104), the processing proceeds to step S106. Specifically, in a case where editing of the key object is disabled in step S104, the key object is not set as the output target.

In step S106, for example, the image capturing control unit 112 sets processing for the next radiological image. After that, the processing returns to step S101. In the re-output processing illustrated in the flowchart of FIG. 9, the processing of steps S101 to S106 is executed on all radiological images included in the examination on which the re-output processing is executed.

As a result of determination in step S101, in a case where there is no unchecked radiological image in the examination targeted for re-output processing (NO in step S101), the image capturing control unit 112 determines that processing has been performed on all radiological images included in the examination targeted for re-output processing, and then the processing proceeds to step S107.

In step S107, the image capturing control unit 112 re-outputs information (the radiological image, the key object, etc.) about the examination added to the output target to the PACS 60 or the like. This prevents the key object for which editing is disabled and which is stored in the PACS 60 or the like from being inadvertently updated from the medical information processing apparatus 10. Then, when the processing of step S107 is finished, the processing in the flowchart illustrated in FIG. 9 is terminated.

In the flowchart illustrated in FIG. 9, the image capturing control unit 112 has a mode for performing control to limit the output of the key object to the storage apparatus, such as the PACS 60, in accordance with the editing enable/disable information 630 that is setting information set in the key object at creation of the key object. The image capturing control unit 112 that performs the control in this mode constitutes an output control unit. In this regard, the present exemplary embodiment is not limited to this mode, and the following modes can also be applied.

For example, the image capturing control unit 112 may have a mode in which the image capturing control unit 112 performs output control for prohibiting output of a key object to the storage apparatus, such as the PACS 60, in a case where a person created the key object set as setting information set in the key object is different from a person editing the key object. In this mode, for example, a key object 600-2 illustrated in FIG. 6B is used as the key object. The key object 600-2 further includes, as setting information, creator information 650 for identifying the creator created the key object 600-2, in addition to the setting information set in the key object 600-1 illustrated in FIG. 6A. In a case where an operator has performed editing including a change of the key object 600-2 via the operation unit 111, for example, the image capturing control unit 112 performs the above-described output control, based on the creator information 650 and editor information identified based on login information or the like about the operator that has performed the editing. In the present exemplary embodiment, the image capturing control unit 112 that performs editing including a change of the key object 600 based on information input by the operator via the operation unit 111 constitutes an editing unit.

Further, for example, the image capturing control unit 112 may have a mode in which the image capturing control unit 112 performs output control for prohibiting output of a key object to the storage apparatus, such as the PACS 60, in a case where a person created the key object set as setting information set in the key object is different from an operator issuing an instruction to perform the above-described re-output processing via the operation unit 111. Also, in this mode, for example, the key object 600-2 illustrated in FIG. 6B is used as the key object. For example, the image capturing control unit 112 may have a mode in which the above-described output control is performed based on the creator information 650 and operator information identified based on login information or the like about the operator in a case where the operator has instructed the above-described re-output processing via the operation unit 111.

Further, for example, the image capturing control unit 112 may have a mode in which the image capturing control unit 112 performs output control for prohibiting output of a key object to the storage apparatus, such as the PACS 60, in a case where the medical information processing apparatus created the key object set as setting information set in the key object is different from the medical information processing apparatus edited the key object. In this mode, for example, a key object 600-3 illustrated in FIG. 6C is used as the key object. This key object 600-3 further includes, as setting information, creation apparatus information 660 for identifying the medical information processing apparatus created the key object 600-3, in addition to the setting information set in the key object 600-1 illustrated in FIG. 6A. For example, in a case where the operator has performed editing including a change of the key object 600-3 via the operation unit 111, the image capturing control unit 112 performs the above-described output control using information about the medical information processing apparatus 10 that has performed the editing and the medical information processing apparatus identified by the creation apparatus information 660.

Further, for example, the image capturing control unit 112 may have a mode in which the image capturing control unit 112 performs output control for prohibiting output of a key object to the storage apparatus, such as the PACS 60, in a case where a classification indicating an intended use of the key object set as setting information set in the key object is not a specific classification. In this mode, for example, the key object 600-1 illustrated in FIG. 6A is used as the key object. The image capturing control unit 112 performs the above-described output control in a case where the classification indicated by the classification information 610 is not a predetermined specific classification (e.g., classifications for “explain to patient” and “follow-up observation” that can be set on the key object creation screen 500 illustrated in FIG. 5).

In the above-described modes, the output control is performed in accordance with setting information set in the key object. However, the present invention is not limited to the modes, and, for example, the following modes can also be applied.

For example, the image capturing control unit 112 may have a mode in which the image capturing control unit 112 performs output control for prohibiting output of a key object to the storage apparatus, such as the PACS 60, in a case where the key object is changed (edited). Further, for example, the image capturing control unit 112 may have a mode in which the image capturing control unit 112 performs output control for prohibiting output of a key object to the storage apparatus, such as the PACS 60, in a case where the medical information processing apparatus 10 is not authenticated (e.g., node authentication) by the storage apparatus such as the PACS 60.

As described above, in the medical information processing apparatus 10 according to the first exemplary embodiment, the image capturing control unit 112 limits output of a key object (e.g., edited key object) to the storage apparatus, such as the PACS 60, in accordance with setting information set in the key object. Further, the image capturing control unit 112 is configured to limit output of a key object to the storage apparatus, such as the PACS 60, in accordance with whether the key object is changed, or whether it is authenticated by the storage apparatus.

According to this configuration, it is possible to prevent a key object, which is related information about a medical image, from being inadvertently updated. Consequently, it is possible to appropriately manage a key object. Specifically, it is possible to prevent a key object from being inadvertently updated, for example, in a case where editing of the key object stored in the storage apparatus, such as the PACS 60, is disabled. It is also possible to prevent misuse of a key object that is not suitable for intended use, or missing of permanent information such as calling for attention on a medical image.

The above-described first exemplary embodiment illustrates a mode in which the medical information processing apparatus 10 acquires the medical image (radiological image) and the related information (key object) stored in association with each other in the PACS 60. However, the present invention is not limited to this mode. For example, a mode in which the medical information processing apparatus 10 acquires a medical image (radiological image) and related information (key object) stored in association with each other in the storage unit of the medical information processing apparatus 10 can also be applied to the present invention. In this mode, the PACS 60 and the storage unit of the medical information processing apparatus 10 constitute a storage unit. Specifically, in this mode, the medical information processing apparatus 10 may have a mode in which the medical information processing apparatus 10 performs output control for limiting output of an edited key object to the storage apparatus, such as the PACS 60, in accordance with setting information set in the key object acquired from the storage unit of the medical information processing apparatus 10, information indicating whether the key object is changed, or information indicating whether it is authenticated by the storage apparatus, such as the PACS 60.

### Second Exemplary Embodiment

Next, a second exemplary embodiment of the present invention will be described. In a description of the second exemplary embodiment to be described below, descriptions of matters common to those of the first exemplary embodiment described above are omitted, and only matters different from those of the first exemplary embodiment will be described.

A schematic configuration of a medical information processing system including a medical information processing apparatus according to the second exemplary embodiment is similar to the schematic configuration of the medical information processing system 1 including the medical information processing apparatus 10 according to the first exemplary embodiment illustrated in FIG. 1.

The above-described first exemplary embodiment illustrates a mode in which output control for limiting the key object to be re-output to the storage apparatus, such as the PACS 60 from the medical information processing apparatus 10, is performed. Meanwhile, in the second exemplary embodiment, the medical information processing apparatus 10 is configured to perform editing control for limiting editing including a change of the created key object.

<Created Key Object Editing Processing>

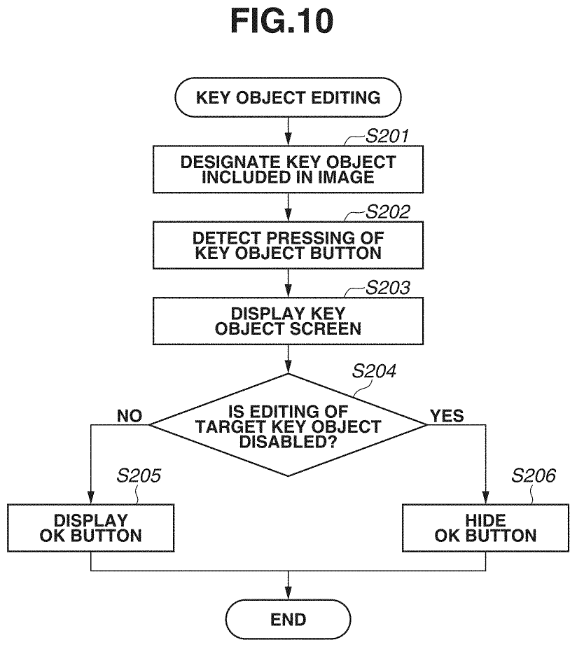

FIG. 10 is a flowchart illustrating an example of a processing procedure for a medical information processing method that is performed by the medical information processing apparatus 10, according to the second exemplary embodiment of the present invention. Specifically, FIG. 10 is a flowchart illustrating an example of a processing procedure for created key object editing processing in the medical information processing apparatus 10.

Prior to the processing in the flowchart illustrated in FIG. 10, the image capturing control unit 112 first performs processing for acquiring various information about the examination targeted for editing from the storage apparatus, such as the PACS 60. Specifically, in this case, the image capturing control unit 112 preliminarily acquires information about the examination, including the radiological image and the key object, targeted for editing from the PACS 60 or the like in which the radiological image, which is a medical image obtained by capturing an image of the subject H. and related information about the radiological image are stored in association with each other. The image capturing control unit 112 that performs this processing constitutes the acquisition unit.

Then, the operator presses the image capturing method button (e.g., the “front of chest” button 251 or the “side of chest” button 252) including the key object to be edited or referred to on the past examination playback screen 800 illustrated in FIG. 8 via the operation unit 111, and plays back the target radiological image on the image display area 410. After that, the processing in the flowchart illustrated in FIG. 10 is started. First, in step S201, for example, the image capturing control unit 112 selects the key object included in the radiological image being played back on the image display area 410 by designating the key object box 413.

After that, for example, in response to the operator pressing the key object button 425 via the operation unit 111, in step S202, for example, the image capturing control unit 112 detects the pressing of the key object button 425.

Next, in step S203, for example, the image capturing control unit 112 displays the key object creation screen 500 for the selected key object on the display unit 115, based on control of the display control unit 114. In this operation, the key object creation screen 500 is displayed on the display unit 115 in a state where an operation on the editing enable/disable setting unit 530 illustrated in FIG. 5 is disabled (or, for example, in a case where “editing disabled” is set, the editing enable/disable setting unit 530 is not displayed).

Next, in step S204, for example, the image capturing control unit 112 determines whether information indicating that editing of the selected key object is disabled is set by the editing enable/disable setting unit 530 at creation of the key object. In this step S204, for example, in a case where the selected key object corresponds to the key object 600-1 illustrated in FIG. 6A, the determination in step S204 may be performed with reference to the editing enable/disable information 630.

As a result of determination in step S204, in a case where editing of the selected key object is not disabled (NO in step S204), the processing proceeds to step S205 to enable editing of the key object.

In step S205, for example, the image capturing control unit 112 displays the OK button 540 to enable editing of the key object, based on control of the display control unit 114.

On the other hand, as a result of determination in step S204, in a case where editing of the selected key object is disabled (YES in step S204), the processing proceeds to step S206 to disable editing of the key object.

In step S206, for example, the image capturing control unit 112 hides the OK button 540 to disable editing of the key object, based on control of the display control unit 114.

Further, the editing enable/disable setting unit 530 is not displayed regardless of the setting. Then, in a case where the processing of step S205 or step S206 is finished, the processing in the flowchart illustrated in FIG. 10 is terminated. The processing in the flowchart illustrated in FIG. 10 inhibits determination processing that is performed by pressing the OK button 540 on the key object for which editing is disabled, on the key object creation screen 500 displayed from the past examination playback screen 800, whereby editing contents cannot be reflected (i.e., editing cannot be performed).

The above-described example illustrates a mode in which display control for the OK button 540 is performed as a method for limiting editing of the key object. However, the present invention is not limited to this mode. For example, the classification setting unit 510 and the annotation designation unit 520 may be displayed in an inoperable state.

In the flowchart illustrated in FIG. 10, the image capturing control unit 112 has a mode in which the image capturing control unit 112 performs control for prohibiting editing of a key object in a case where information indicating that editing of the key object is disabled is set, as an example of limiting editing in accordance with setting information set in the key object. The image capturing control unit 112 that performs the control in this mode constitutes an editing control unit. In this regard, the present exemplary embodiment is not limited to this mode, and the following modes can also be applied.

For example, the image capturing control unit 112 may have a mode in which the image capturing control unit 112 performs editing control for prohibiting editing of a key object in a case where a person created the key object set as the setting information set in the key object is different from a person edited the key object. In this mode, for example, the key object 600-2 illustrated in FIG. 6B is used as the key object. In a case where the operator performs editing including a change of the key object 600-2 via the operation unit 111, the image capturing control unit 112 performs the above-described editing control, based on the creator information 650 and the editor information identified based on login information or the like about the operator performing the editing. In the present exemplary embodiment, the image capturing control unit 112 that performs editing including a change of the key object 600, based on information input by the operator via the operation unit 111, constitutes the editing unit.

Further, for example, the image capturing control unit 112 may have a mode in which the image capturing control unit 112 performs editing control for prohibiting editing of a key object in a case where a person created the key object set as the setting information set in the key object is different from the operator issuing an instruction to perform the re-output processing, described in the first exemplary embodiment, via the operation unit 111. Also, in this mode, for example, the key object 600-2 illustrated in FIG. 6B is used as the key object. The image capturing control unit 112 may have a mode in which the image capturing control unit 112 performs the above-described editing control based on the creator information 650 and the operator information identified based on login information or the like about the operator, for example, in a case where the operator instructs to perform the above-described re-output processing via the operation unit 111.

Further, for example, the image capturing control unit 112 may have a mode in which the image capturing control unit 112 performs editing control for prohibiting editing of a key object is performed in a case where the medical information processing apparatus created the key object set as the setting information set in the key object is different from the medical information processing apparatus edited the key object. In this mode, for example, the key object 600-3 illustrated in FIG. 6C is used as the key object. The image capturing control unit 112 performs the above-described editing control using the medical information processing apparatus 10 that performs the editing and the medical information processing apparatus identified by the creation apparatus information 660, for example, in a case where the operator performs editing including a change of the key object 600-3 via the operation unit 111.

Further, for example, the image capturing control unit 112 may have a mode in which the image capturing control unit 112 performs editing control for prohibiting editing of a key object in a case where a classification indicating an intended use of the key object set as the setting information set in the key object is not a specific classification. In this mode, for example, the key object 600-1 illustrated in FIG. 6A is used as the key object. The image capturing control unit 112 performs the above-described editing control in a case where the classification indicated by the classification information 610 is not a predetermined specific classification (e.g., classifications for “explain to patient” and “follow-up observation” that can be set on the key object creation screen 500 illustrated in FIG. 5).

As described above, in the medical information processing apparatus 10 according to the second exemplary embodiment, the image capturing control unit 112 limits editing including a change of the key object in accordance with setting information set in the key object.

According to this configuration, it is possible to prevent the key object, which is related information about a medical image, from being inadvertently updated. Consequently, it is possible to appropriately manage the key object. Specifically, for example, in a case where editing of the key object stored in the storage apparatus such as the PACS 60 is disabled, the key object is prevented from being inadvertently updated.

The above-described second exemplary embodiment illustrates a mode in which the medical information processing apparatus 10 acquires the medical image (radiological image) and the related information (key object) stored in association with each other in the PACS 60. However, the present invention is not limited to this mode. For example, a mode in which the medical information processing apparatus 10 acquires a medical image (radiological image) and related information (key object) stored in association with each other in the storage unit of the medical information processing apparatus 10 can be applied to the present invention. Further, in this mode, the storage unit (the PACS 60 may also be included) of the medical information processing apparatus 10 constitutes the storage unit. Specifically, in this mode, the medical information processing apparatus 10 may have a mode in which editing control for limiting editing including a change of the key object is performed in accordance with setting information set in the key object acquired from the storage unit of the medical information processing apparatus 10.

### Other Exemplary Embodiments

The present invention can also be implemented by processing in which a program for implementing one or more functions according to the above-described exemplary embodiments is supplied to a system or an apparatus via a network or a storage medium, and one or more processors in a computer of the system or the apparatus read out and execute the program. The present invention can also be implemented by a circuit (e.g., an application specific integrated circuit (ASIC)) for implementing one or more functions.

The above-described exemplary embodiments of the present invention are merely embodiments for carrying out the present invention, and the technical scope of the present invention should not be limitedly interpreted by the exemplary embodiments. That is, the present invention can be carried out in various forms without departing from the technical idea or principal features of the present invention.

The present invention is not limited to the above-described exemplary embodiments, and can be modified or changed in various ways without departing from the spirit and scope of the present invention. Therefore, the following claims are attached to disclose the scope of the present invention.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

According to the present invention, it is possible to prevent related information about a medical image from being inadvertently updated. Consequently, it is possible to appropriately manage the related information.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A medical information processing system comprising:
a storage unit configured to store a medical image obtained by capturing an image of a subject and related information about the medical image in association with each other; and
an editing control unit configured to limit editing including a change of the related information stored in the storage unit, in accordance with setting information set in the related information.

2. The medical information processing system according to claim 1, further comprising:
an acquisition unit configured to acquire the medical image and the related information associated with each other,
wherein the editing control unit limits the editing including a change of the related information acquired by the acquisition unit.

3. The medical information processing system according to claim 1, wherein the editing control unit prohibits the editing of the related information in a case where editing of the related information is disabled as the setting information set in the related information.

4. The medical information processing system according to claim 1, wherein the editing control unit prohibits the editing of the related information in a case where a person created the related information set as the setting information set in the related information is different from a person editing the related information.

5. The medical information processing system according to claim 1, wherein the editing control unit prohibits the editing of the related information in a case where a person created the related information set as the setting information set in the related information is different from an operator issuing an instruction to output the medical image and the related information associated with each other to the storage unit via an operation unit.

6. The medical information processing system according to claim 1, wherein the editing control unit prohibits the editing of the related information in a case where a medical information processing apparatus performing the editing of the related information is different from a medical information processing apparatus created the related information set as the setting information set in the related information.

7. The medical information processing system according to claim 1, wherein the editing control unit prohibits the editing of the related information in a case where a classification indicating an intended use of the related information set as the setting information in the related information is not a specific classification.

8. A medical information processing system comprising:
a storage unit configured to store a medical image obtained by capturing an image of a subject and related information about the medical image in association with each other;
an acquisition unit configured to acquire the medical image and the related information associated with each other;
an editing unit configured to edit the related information acquired by the acquisition unit; and
an output control unit configured to limit an output of the edited related information to the storage unit, in accordance with setting information set in the related information.

9. The medical information processing system according to claim 8, wherein the output control unit prohibits the output of the related information to the storage unit in a case where editing of the related information is disabled as the setting information set in the related information.

10. The medical information processing system according to claim 8, wherein the output control unit prohibits the output of the related information to the storage unit in a case where a person created the related information set as the setting information in the related information is different from a person editing the related information.

11. The medical information processing system according to claim 8, wherein the output control unit prohibits the output of the related information to the storage unit in a case where a person created the related information set as the setting information set in the related information is different from an operator issuing an instruction for the output via an operation unit.

12. The medical information processing system according to claim 8, wherein the output control unit prohibits the output of the related information to the storage unit in a case where a medical information processing apparatus created the related information set as the setting information set in the related information is different from a medical information processing apparatus edited the related information.

13. The medical information processing system according to claim 8, wherein the output control unit prohibits the output of the related information to the storage unit in a case where a classification indicating an intended use of the related information set as the setting information set in the related information is not a specific classification.

14. The medical information processing system according to claim 8, wherein the output control unit prohibits the output of the related information to the storage unit in a case where the related information is changed.

15. The medical information processing system according to claim 8, wherein the output control unit prohibits the output of the related information to the storage unit in a case where a medical information processing apparatus edited the related information is not authenticated by the storage unit.

16. A medical information processing method comprising:
storing a medical image obtained by capturing an image of a subject and related information about the medical image in association with each other in a storage unit; and
performing editing control of limiting editing including a change of the related information stored in the storage unit, in accordance with setting information set in the related information.

17. A medical information processing method comprising:
storing a medical image obtained by capturing an image of a subject and related information about the medical image in association with each other in a storage unit;
acquiring the medical image and the related information associated with each other;
editing the related information acquired in the acquiring; and
performing output control of limiting an output of the edited related information to the storage unit, in accordance with setting information set in the related information.

18. A storage medium storing a program for executing a medical information processing method comprising:
storing a medical image obtained by capturing an image of a subject and related information about the medical image in association with each other in a storage unit; and
performing editing control of limiting editing including a change of the related information stored in the storage unit, in accordance with setting information set in the related information.

19. A storage medium storing a program for executing a medical information processing method comprising:
storing a medical image obtained by capturing an image of a subject and related information about the medical image in association with each other in a storage unit,
acquiring the medical image and the related information associated with each other;
editing the related information acquired in the acquiring; and
performing output control of limiting an output of the edited related information to the storage unit, in accordance with setting information set in the related information.
